(19) United States
(12) Patent Application Publication
(10) Pub. No.: US 2006/0235418 A1
(43) Pub. Date: Oct. 19, 2006

(54) Method and device for preparing a surface for receiving an implant

(75) Inventors: Carlos Gil, Collierville, TN (US); Randall Allard, Germantown, TN (US); Greg Marik, Germantown, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/105,219

(22) Filed: Apr. 13, 2005

(51) Int. Cl. A61B 17/00

(52) U.S. Cl. 606/79

(57) ABSTRACT

Methods for forming a shaped surface, for example, a shaped end plate between adjacent vertebral bodies, and a bone removal device for use in such methods. The device includes a contour having a predetermined profile corresponding at least in part to an implant, and a cutting element following the contour and having a cutting edge having a profile corresponding to the contour profile.

### BACKGROUND

The present disclosure relates generally to devices and methods for preparing a surface having a shape that corresponds at least in part to the shape of an implant to be inserted into contact with the surface. According to an exemplary embodiment, shaped endplates between adjacent vertebral bodies are prepared to receive an implant of a corresponding shape.

Present methods of forming an implantation space between adjacent vertebral bodies in the human spine generally include the use of one or more of the following: rongeurs, curettes, mills and chisels. Forming an implantation space so as to provide a surface shape that closely matches the shape of the implant provides for adequate support surface across which the load transfer between the adjacent surfaces can be evenly applied. In instances where the surface that the implant will come into contact with has not been shaped to correspond to the shape of the implant, the implant may slip, or be forcefully ejected from the space between the adjacent vertebral bodies, or lacking broad contact between the implant and the vertebral bodies, a failure to obtain fusion may occur.

### SUMMARY

The present invention relates to methods for preparing a surface, for example, a shaped end plate between adjacent vertebral bodies, and a bone removal device for use in such methods.

In an exemplary embodiment, a bone removal device and associated method are adapted to form a surface on or into one or more of the vertebral body surfaces that are adjacent the intervertebral disc space. The formed surface(s) have a defined shape corresponding at least in part to that of a selected interbody spinal implant to be implanted in the disc space.

According to one example, the device comprises a shaft, a contour attached to the shaft, and a cutting element attached to the contour. The contour has a predetermined profile, which may correspond, at least in part, to the profile of a selected implant. The cutting element has a cutting edge, which cutting edge has a profile corresponding to the contour profile.

According to another example, a method of preparing a space between first and second vertebral bodies to receive an implant is provided. The method includes attaching first and second anchoring devices to the first and second vertebral bodies, respectively; attaching a distraction assembly to the first and second anchoring devices; attaching a bone removal device to the first distractor arm; and shaping a first endplate of the first vertebral body with the bone removal device. The distractor assembly has a first arm attached to the first anchoring device and a second arm attached to the second anchoring device. The bone removal device comprises a contour and a cutting element attached to the contour, which contour has a profile corresponding at least in part to the profile of the implant, and which cutting element has a cutting edge having a profile corresponding to the contour profile.

According to another exemplary method for preparing a site between first and second vertebral bodies to receive an implant, space is created between first and second vertebral bodies. The space created is sufficient to allow access to the disc space between the vertebral bodies for the use of a bone removal device. According to such a method, endplates of the vertebral bodies are shaped with a bone removal device comprising a contour and a cutting element attached to the contour. The contour has a profile corresponding at least in part to the profile of the implant, and the cutting element has a cutting edge having a profile corresponding to the contour profile. In some examples of such a method, space between the first and second vertebral body is created with a distraction assembly.

According to another example, a second endplate of the second vertebral body is shaped with a bone removal device having the same or different contour and cutting element as those used to shape the first vertebral body.

According to still other examples, a system for preparing a site is provided. The system comprises an access instrument operable to provide access to the site, and a bone removal device. The bone removal device comprises a contour and a cutting element attached to the contour, which contour has a profile corresponding at least in part to the profile of an implant to be inserted at the site, and which cutting element has a cutting edge having a profile corresponding to the contour profile.

### BRIEF DESCRIPTION OF DRAWINGS

The disclosure can be more clearly understood by reference to the following drawings, which illustrate exemplary embodiments thereof, and which are not intended to limit the scope of the appended claims.

FIG. 1A illustrates an exemplary device for preparing a site to receive an implant.

FIGS. 1B and 1C illustrate an exemplary method for forming the cutting element of the exemplary device illustrated in FIG. 1A.

FIG. 2A illustrates an exemplary alternative device for preparing a site to receive an implant.

FIG. 2B illustrates another exemplary alternative device for preparing a site to receive an implant.

FIG. 3 illustrates a vertebral column having a damaged disc.

FIG. 4 illustrates an exemplary access instrument for use with the device of FIG. 1A.

FIG. 5 illustrates an anchoring device for use with the exemplary access instrument illustrated in FIG. 4.

FIG. 6 illustrates an anchoring device for use with the exemplary access instrument illustrated in FIG. 4.

FIG. 7 illustrates the anchoring devices illustrated in FIGS. 5 and 6 attached to the exemplary access instrument illustrated in FIG. 4.

FIGS. 8A and 8B illustrate manipulation of the anchoring devices illustrated in FIG. 7.

FIG. 9 illustrates an exemplary alignment guide for use with the exemplary access instrument illustrated in FIG. 4.

FIG. 10 illustrates the exemplary alignment guide illustrated in FIG. 9 attached to the access instrument illustrated in FIG. 4.

FIG. 11 is an exploded view of an exemplary device for preparing a site to receive an implant.

FIG. 12 illustrates use of the exemplary device illustrated in FIG. 11 to prepare a vertebral endplate.

The disclosure can be more clearly understood by reference to some of its specific embodiments, described in detail below, which description is not intended to limit the scope of the claims in any way.

### DETAILED DESCRIPTION

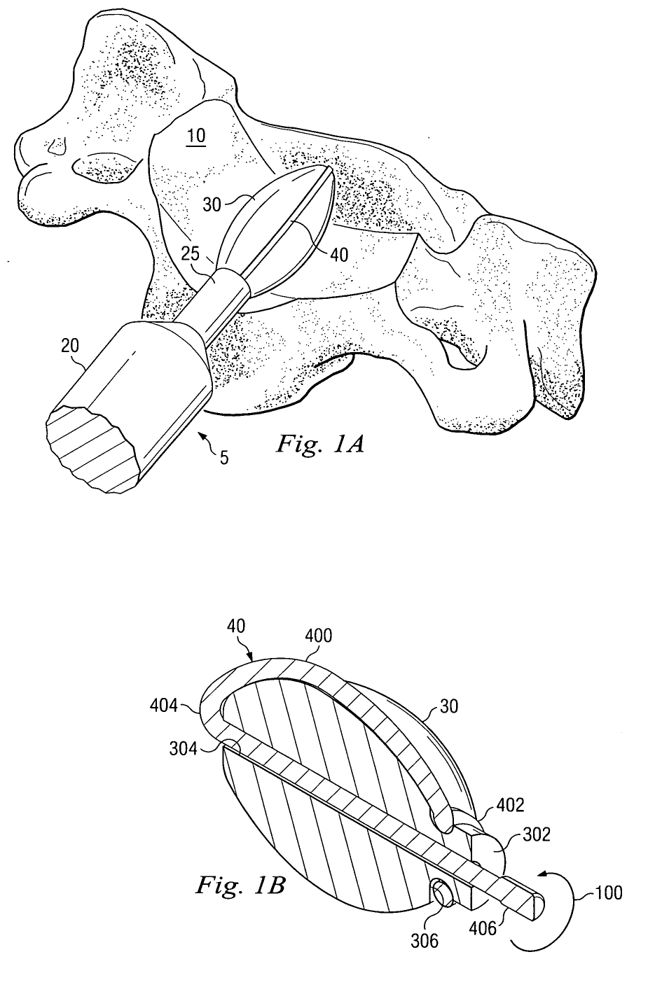

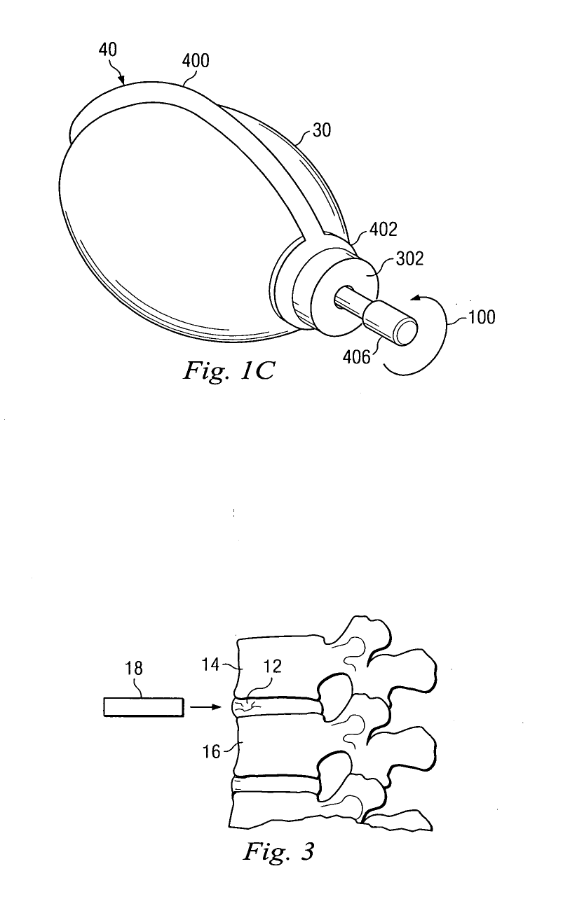

Referring now to FIG. 1A, an exemplary bone removal device 5 is illustrated adjacent a vertebral endplate 10 to be shaped. Bone removal device 5 includes an exterior shaft 20, an interior shaft 25, a contour 30, and a cutting element 40. The cutting element 40 is rotatably engaged with the contour 30 so that the contour remains stationary with respect to the cutting element 40, or alternatively, to a point on the cutting element, while the cutting element 40 rotates around the contour 30. The cutting element 40 has a cutting edge 400 that shapes the endplate 10 as the cutting edge 400 comes into contact with the endplate.

In certain examples, cutting element 40 comprises a wire attached to the contour. In examples where the cutting element comprises a wire, the wire can be made from any material having suitable strength for cutting a hard surface, such as bone or a vertebral endplate. Exemplary materials include but are not limited to nitinol, a synthetic polymer cable, a braided wire cable, stainless steel, titanium alloy, a plastic having a tensile strength great enough to allow the cutting element to shape a surface, polyester, polyethylene, and a variety of commercially available polymers, such as PEEK™ polymer, which is commercially available from Invibio, Inc.

According to other examples, cutting element 40 comprises a metal, and the cutting edge 400 comprises a thin blade of the metal. Exemplary metals for forming such a cutting element include but are not limited to stainless steel and titanium alloys.

FIGS. 1B and 1C illustrate an exemplary method for rotatably securing a cutting element 40 comprising a wire to a contour 30. In the example illustrated in FIGS. 1B and 1C, contour 30 comprises a neck 302 terminating the contour at a distal end, and a bore 304 extending through the interior of the contour, and having an exit at the distal end of the contour through the neck 302. The contour 30 further comprises an annular recess 306, extending circumferentially around the neck 302.

The cutting element 40 comprises a cutting edge 400, a neck mating portion 402 and a bore mating portion 404. The neck mating portion 402 rotatably resides within the annular recess 306. The bore mating portion 404 extends through the bore 304, and exits the bore at the neck 302. The bore mating portion 404 rotatably resides within the bore 304. The bore mating portion 404 terminates in a connecting portion 406, which can be connected to means for causing the rotation of neck mating portion 402 and bore mating portion 404, for example in a counterclockwise direction 100. As the neck mating portion 402 rotates within the annular recess 306 and the bore mating portion 404 rotates within the bore 304, the cutting edge 400 consequently rotates around the contour 30, in the same direction of rotation as the neck mating portion 402 and the bore mating portion 404.

The rotation of the cutting edge 400, neck mating portion 402, bore mating portion 404 and connecting portion 406 can be in a clockwise or counterclockwise direction 100, as illustrated in FIG. 1B, and can be a 360° or 180° rotation, or any degree of rotation therebetween, or even less than 180°.

In use, the bone removal device 5 is operably connected to a power source (not shown), which may be any conventional power source such as an electric or air-powered motor. For example, the power source can actuate the interior shaft 25, which itself can be connected to a connecting portion of the cutting element, for example, connecting portion 406 illustrated in FIGS. 1B and 1C. Rotation of the connecting portion 406 is coincidental with, and is along the same axis of rotation as, the interior shaft 25. Actuating the connecting portion for rotation also actuates the remaining portions of the cutting element 40, for example, neck mating and bore mating portions. As discussed above with respect to FIGS. 1B and 1C, rotation of the connecting, neck mating, and bore mating portions of the cutting element 40 causes rotation of the cutting edge 400, thereby causing the cutting element 40 to shape the vertebral endplate 10.

Cutting element 40 has a cutting edge 400 having the same profile as the contour 30. Thus, as the cutting edge 400 comes into contact with a surface, the cutting element 40 will shape the surface, for example, an endplate of a vertebral body, to have a profile corresponding to the contour 30. In turn, the contour 30 can be shaped to correspond at least in part to that of an implant to be inserted between vertebral bodies.

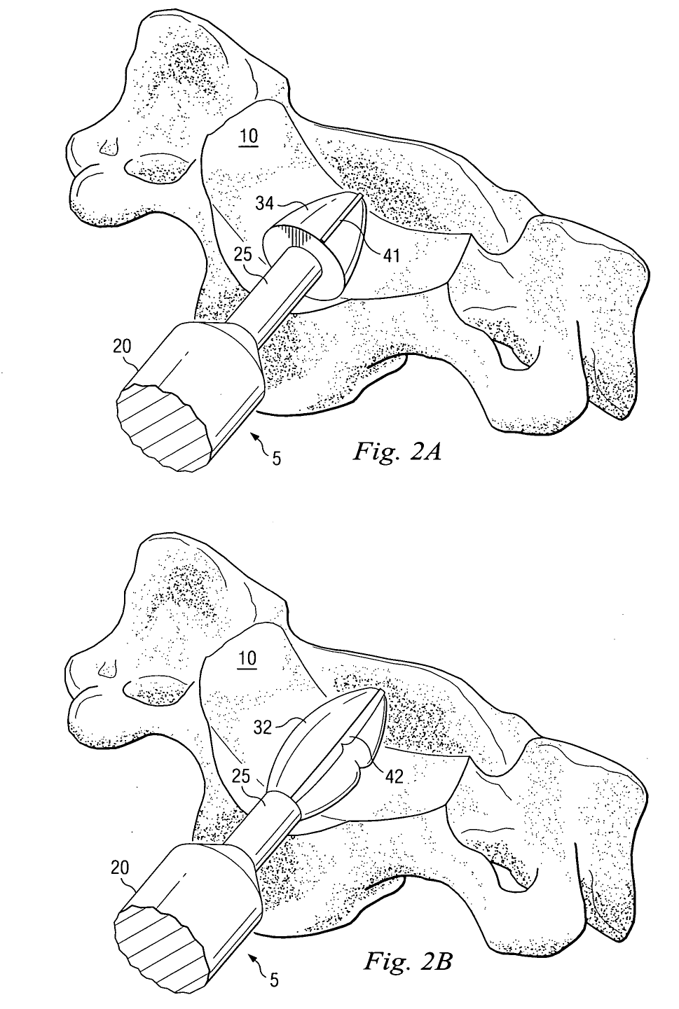

While contour 30 illustrated in FIG. 1A is egg-shaped, contour 30 can be designed to have any shape and/or size suitable for corresponding in at least some respect to the shape and/or size of implant to be inserted. For example, if the profile of the implant to be inserted has a parabolic aspect, or a double-hump aspect, then the contour could be shaped to correspond to that aspect. The cutting edge of the cutting element would be shaped to correspond to the shape of the contour. For example, FIG. 2A illustrates a contour 34 having a parabolic shape and a cutting element 41 having a parabolic shape at its cutting edge. FIG. 2B illustrates a contour 32 having a double-hump shape, and cutting element 42 having a double-hump shape at its cutting edge. Thus, the contour need only have a predetermined profile corresponding at least in part to the profile of the particular implant, and the cutting edge of the cutting element corresponds to the profile of the contour. In addition, contour 30 can be interchangeable, such that it can be removed from the shaft 25, and replaced with a contour of the same or a different shape or size.

Although a bone removal device is illustrated in FIGS. 1A-1C and 2A-2B for use in preparation of a vertebral endplate for an implant, bone removal devices as described herein can be used in processes for repairing any damaged joint where it is desirable to prepare a surface having a given shape.

An exemplary use of a bone removal device as described herein for repairing a damaged joint is illustrated in FIGS. 3-12. In particular, FIGS. 3-12 illustrate the replacement of an injured, diseased, or otherwise damaged intervertebral disc 12 extending between adjacent vertebrae 14, 16. The damaged disc may be replaced by an intervertebral disc prosthesis or fusion device 18 which may be a variety of devices, including the prostheses which have been described in U.S. Pat. Nos. 5,674,296; 5,865,846; 6,156,067; 6,001,130, each of which is incorporated by reference herein, and in U.S. Patent Application Publication Nos. 2002/0035400; 2002/0128715; and 2003/0135277, each of which is incorporated by reference herein.

In methods for inserting an implant between vertebrae 14, 16, all or a portion of the damaged disc 12 is excised. This procedure may be performed using an anterior, anterolateral, lateral, or other approach known to one skilled in the art, however, the following embodiments will be directed toward a generally anterior approach. Generally, a tissue removal procedure may include positioning and stabilizing the patient, and may include a discectomy procedure. The tissue surrounding the disc space may be retracted to access and verify the target disc space. The area of the target disc may be prepared by removing excess bone, including osteophytes which may have developed, and other tissues which may include portions of the annulus and all or portions of the nucleus pulpous. Alignment and/or measurement procedures may precede or follow tissue removal.

According to an exemplary embodiment, an access instrument is inserted into the disc space at a point after tissue removal to provide access to the site to be prepared for implant insertion. In certain examples, the access provided to the implant site is that amount that is sufficient to allow a bone removal device to be manipulated within the disc space between the vertebrae where the implant is to be inserted. Any device capable of providing space between first and second vertebral bodies sufficient to provide access to the site into which an implant will be inserted is suitable. Exemplary devices include but are not limited to distractors, spreaders, and other devices known to those of ordinary skill in the art.

In certain examples, once sufficient space is created between the vertebral bodies, a bone removal device as described herein is manipulated within the disc space so as to shape the endplates of the vertebral bodies. The bone removal device can be manipulated by hand, or can be secured to some type of anchoring or alignment device, including the instrument used to provide access to the disc space.

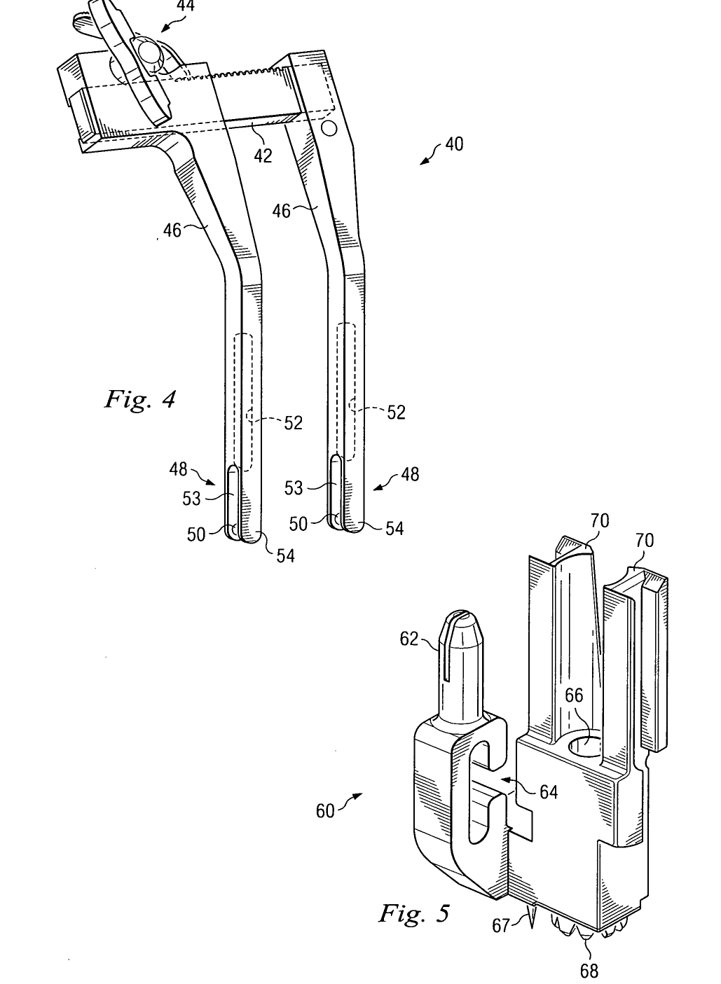

Referring now to FIG. 4, an exemplary access instrument, specifically, a distractor assembly 40, is illustrated. Distractor assembly 40 includes a cross bar member 42 having a securing mechanism 44. A pair of distracting arms 46 is attached to the cross bar member 42. A variety of securing mechanisms 44 may be used to maintain a selected distance between the distracting arms 46 including a ratchet system, clamps, threaded connectors, pins, gripping hardware, or other fasteners. At least one of the distracting arms 46 may be movably connected to cross bar member 42 with the securing mechanism 44.

In the example illustrated in FIG. 4, the distracting arms 46 have curved end portions 54. In other examples, the end portions 54 may be angled or relatively flat.

Each of the distracting arms 46 includes attachment mechanisms 48. In the embodiment of FIG. 4, the attachment mechanisms 48 includes pins 50 and hollow recesses 52. In some embodiments, as shown, one or more of the walls of the hollow recesses 52 have elongated openings 53. The attachment mechanisms 48 may be used to locate, hold, and/or guide one or more anchoring devices as will be described further with respect to FIGS. 5-7. The attachment mechanisms 48 may also include stops or other features useful for position verification or instrument support.

Referring now to FIG. 5, an anchoring device 60 includes a connecting portion 62, a pivot mechanism 64, a vertebral body attachment portion 66, a restraint pin 67, a seat 68, and constraint members 70. The anchoring device 60 can be attached to one of the distracting arms 46 by engaging the pin 50 with the pivot mechanism 64 and by inserting the connecting portion 62 into one of the hollow recesses 52. Restraint pin 67 can be retractable or fixed.

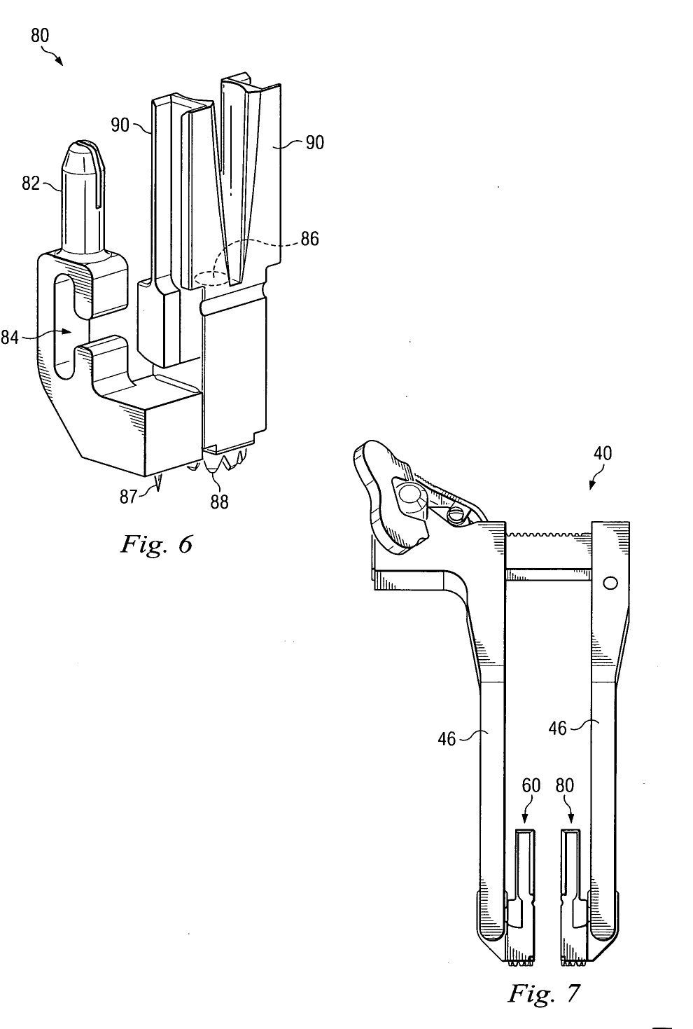

Referring now to FIG. 6, an anchoring device 80, which may be complementary to the anchoring device 60, includes a connecting portion 82, a pivot mechanism 84, a vertebral body attachment portion 86, a restraint pin 87, a seat 88, and constraint members 90. The anchoring device 80 can be attached to one of the distracting arms 46 by engaging the pin 50 with the pivot mechanism 84 and by inserting the connecting portion 82 into one of the hollow recesses 52. Restraint pin 87 can be retractable or fixed. In some embodiments, the anchoring devices 60, 80 may be identical rather than complementary.

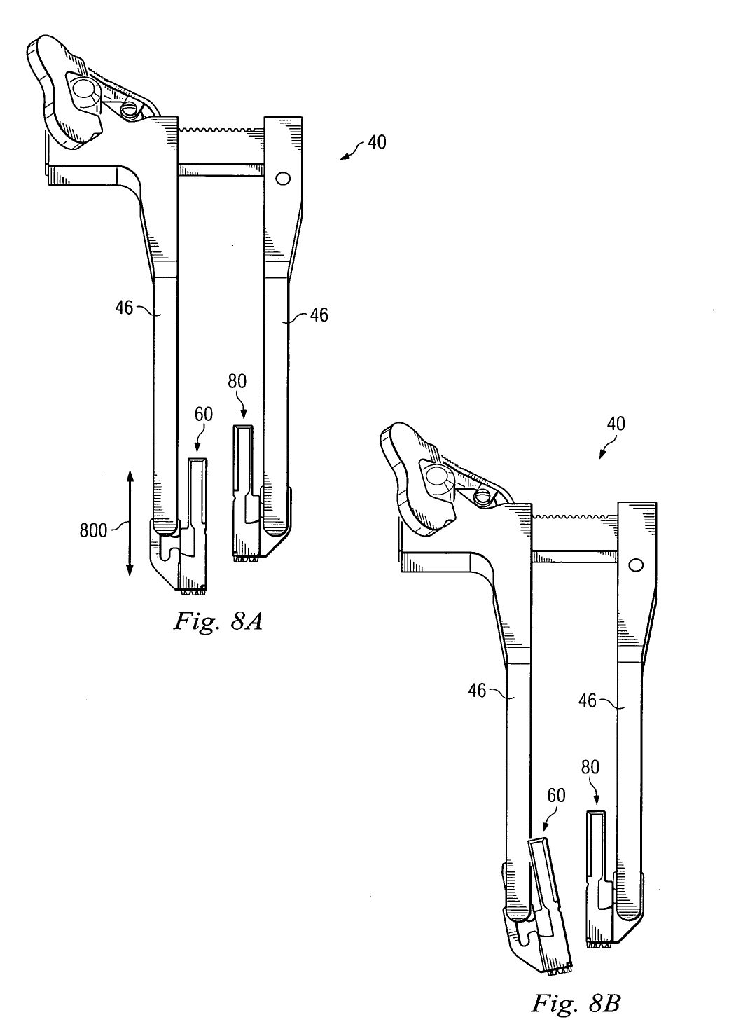

In the exemplary embodiments illustrated in FIGS. 5 and 6, pivot mechanisms 64, 84 are “C”-shaped, which allows for independent displacement of the anchoring devices 60, 80 relative to one another, which will be discussed further with respect to FIGS. 7, 8a and 8b. In addition, the anchoring devices can be moved in a sagittal plane, a transverse plane, with pivotal motion, or linearly in an anterior-posterior direction.

FIG. 7 illustrates a distractor assembly 40 with both anchoring devices 60, 80 attached to distractor arms 46. In other examples, only one of anchoring devices 60, 80 is attached to a distractor arm 46. One or more anchoring devices 60, 80 may be used to located, hold, guide, and/or manipulate subsequent instrumentation.

FIG. 8a illustrates independent manipulation of the anchoring devices 60, 80 relative to one another along an axis 800 aligned with the axis of the hollow recess 52. When using an anterior surgical technique, the axis 800 may be an anterior-posterior axis. FIG. 8b illustrates independent pivoting or rotation of the anchoring devices 60, 80 in a sagittal plane about the pins 50. In this embodiment, the connecting portions 62, 82 may be pulled from the hollow recesses 52. As the anchoring devices 60, 80 pivot independently of each other, the connecting portions 62, 82 may be permitted to pivot in and out of the elongated openings 53 of the distracting arms 46.

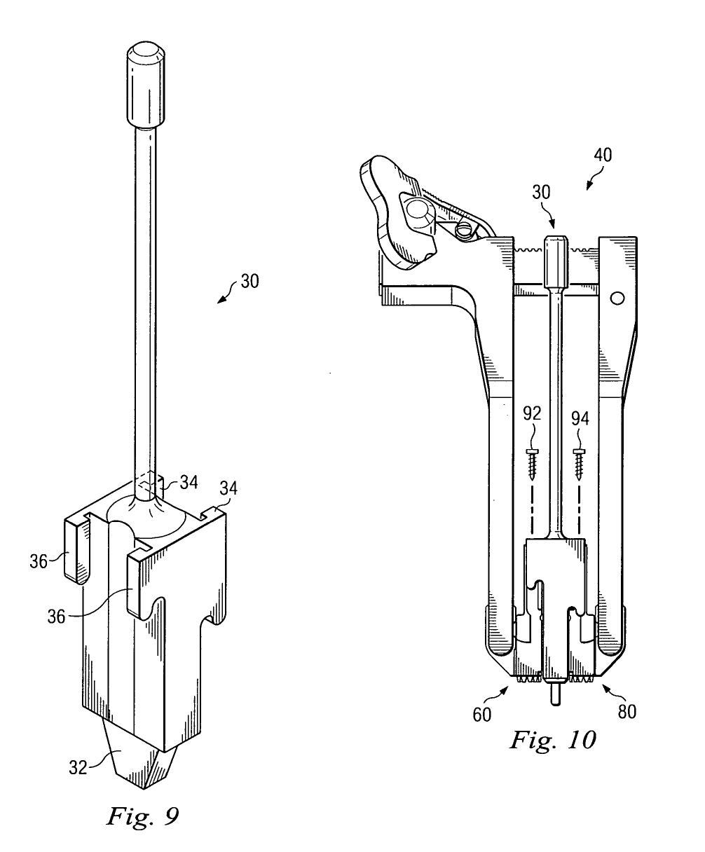

Referring now to FIG. 9, an example of a tool suitable for coupling to anchoring devices 60, 80, specifically an alignment guide 30, is illustrated. Alignment guide 30 comprises an intervertebral portion 32 and positioning guides 34, 36. In the exemplary embodiment illustrated in FIG. 9, the positioning guides 34, 36 have differing lengths to facilitate coupling to subsequent instrumentation, such as anchoring devices 60, 80 and/or distractor assembly 40.

Referring now to FIG. 10, the alignment guide 30 is illustrated coupled to the anchoring devices 60, 80. Specifically, in the illustrated embodiment, one set of positioning guides, for example guides 34, mates with the constraint portions 90. Then, the second set of positioning guides 36 mates with the constraint portions 70. The differing lengths of the positioning guides 34, 36 may allow the surgeon to more easily align the positioning guides with the constraint portions. The constraint portions 70, 90 may prevent movement of the alignment guide 30 relative to the anchoring devices 60, 80, respectively.

With the alignment guide 30 coupled to the anchoring devices 60, 80, the intervertebral portion 32 is inserted between the vertebral endplates of vertebral bodies 14, 16. Alternatively, the insertion of intervertebral portion 32 between the vertebral endplates takes place before or as the alignment guide 30 is coupled to the anchoring devices 60, 80.

The anchoring devices 60, 80 may be positioned equidistant from the mid-line center of the intervertebral disc space. Mid-line alignment of the alignment guide 30 may be confirmed, and the sagittal placement of the alignment guide 30 may be assessed with fluoroscopic or other imaging techniques. After alignment has been assessed, the alignment guide 30 may be locked in place to either or both of the distractor assembly 40 and the anchoring devices 60, 80. During these alignment procedures, the alignment guide 30 may be generally parallel to the plane of the intervertebral disc space.

With the alignment verified, a hole is drilled into the caudal vertebral body 16 through the vertebral body attachment portion 66 of the anchoring device 60. An anchoring fixture 92, such as a bone screw, is inserted through the vertebral body attachment portion 66 and into the vertebral body 16, thus locking the seat 68 to the vertebral body 16. As the anchoring fixture 92 descends through the vertebral body attachment portion 66, the anchoring fixture 92 pushes on the retractable restraint pin 67, embedding the pin 67 in the vertebral body 16 to prevent rotation of the anchoring device 60 and the subsequent loosening of the anchoring fixture 60 from the vertebral body 16.

The seats 68, 88 of the anchoring devices 60, 80, respectively, are adjustable and thus may be raised, lowered, and/or tilted. With the seat 68 locked to the vertebral body 16, the seat 88 of the cephalad anchoring device 80 may be adjusted to contact the vertebral body 14, maintaining the alignment guide 30 aligned in a generally anterior-posterior direction. The seat 88 may be adjusted to level the anchoring devices 60, 80, using for example a bubble level (not shown).

With the seat 88 in position, a second hole is drilled into the cephalad vertebral body 14 through the vertebral body attachment portion 86 of the anchoring device 80. Another anchoring fixture 94, such as a bone screw, is inserted through the vertebral body attachment portion 86 and into the vertebral body 14, thus locking the seat 88 to the vertebral body 14. As the anchoring fixture 94 descends through the vertebral body attachment portion 86, the anchoring fixture 94 pushes on the retractable restraint pin 87, embedding the pin 87 in the vertebral body 14 to prevent rotation of the anchoring device 80 the subsequent loosening of the anchoring fixture 80 from the vertebral body 14. It is understood that in an alternative embodiment, the cephalad anchoring fixture 94 may be placed before the caudal anchoring fixture 92. With the anchoring fixtures 92, 94 in place, the alignment guide 30 may be removed.

With the distractor arms 46 attached to the vertebral bodies 14, 16 by the anchoring devices 80, 60 respectively, the arms 46 may be moved apart, thus placing the vertebral bodies 14, 16 in tension and providing access to the intervertebral space to allow further discectomy and/or decompression procedures as needed. During the distraction, the distractor arms 46 may remain relatively parallel. The securing mechanism 44 may be applied to maintain the vertebral bodies 14, 16 in the desired distracted position.

As the distraction is performed, the connecting portions 62, 82 may remain inside the hollow recesses 52 thereby causing the adjacent endplates of vertebral bodies 14, 16 to remain relatively parallel. Alternatively, during distraction, the connecting portions 62, 82 may be pulled from the hollow recesses 52, and the anchoring devices 60, 80 may pivot about pins 50 (as described above) allowing independent movement of the vertebral bodies 14, 16. In some embodiments, the rotation of the vertebral bodies 14, 16 may be constrained to a transversely centered sagittal plane. In other embodiments, the vertebral bodies 14, 16 may rotate in parallel sagittal planes. Such independent movement is one example of a method that permits independent preparation of the endplates of vertebral bodies 14, 16.

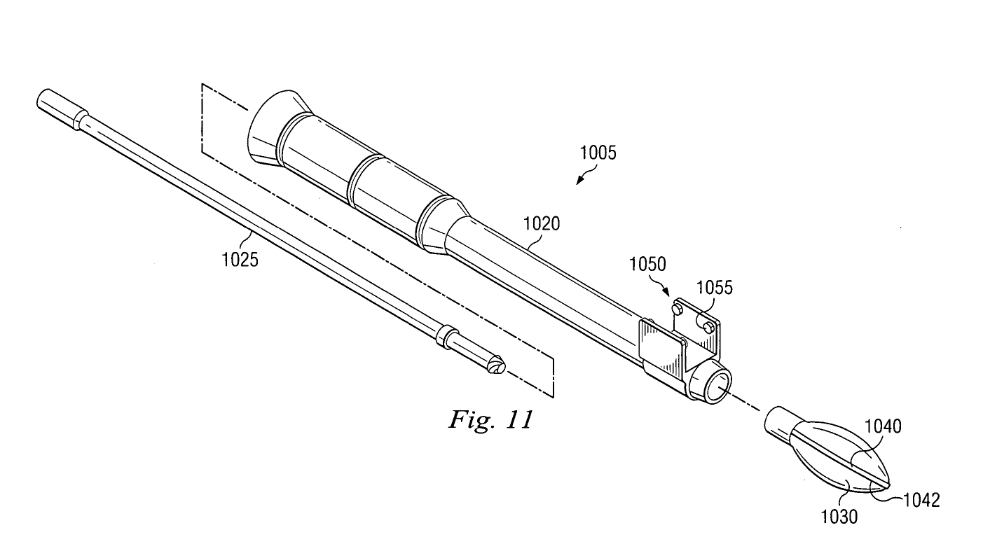

Referring now to FIG. 11, an example of a bone removal device as described herein for use in preparation of the endplate surfaces for placement of an intervertebral prosthesis is illustrated. In the exemplary embodiment illustrated in FIG. 11, the bone removal device 1005 comprises an exterior shaft 1020, an interior shaft 1025, a contour 1030, and a cutting element 1040.

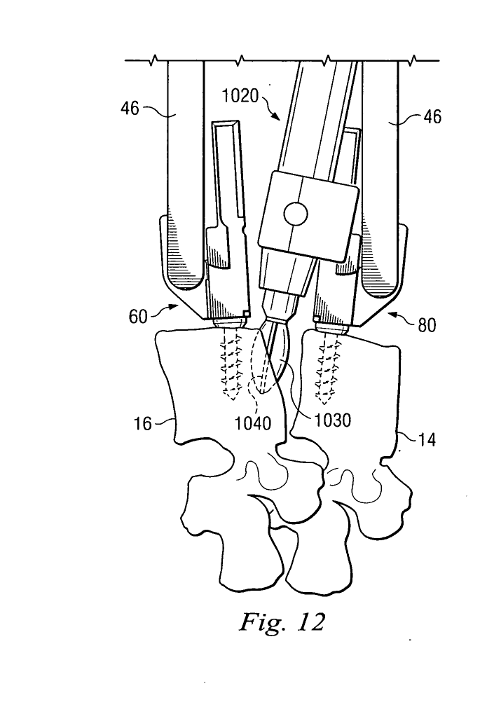

Bone removal device 1005 also includes a coupling 1050, which has fasteners 1055 for attaching the bone removal device 1005 to an anchoring device attached to a distractor assembly, as will be discussed further with respect to FIG. 12. Coupling 1050 can be permanently fixed, such as by welding, or removably fixed, such as by sliding or clipping, to the exterior shaft 1020.

The internal shaft 1025 extends through the exterior shaft 1020 to engage the contour 1030. The cutting element 1040 is attached to the contour 1030. The bone removal device 1005 may include a variety of other components (not shown) such as rivets, bearings, gears, and springs which may be used to assemble the exterior shaft, interior shaft, contour and cutting element to each other and provide movement to the contour and cutting element. Those of ordinary skill in the art can select appropriate components for assembling the parts of a bone removal device as described herein through routine experimentation.

Contour 1030 and cutting element 1040 are selected such that the contour and a cutting edge of the cutting element have a profile corresponding at least in part to the profile of an implant to be inserted. The size of the implant, and measurements, if any, taken of the implant space can guide the selection of the contour and cutting element. The contour 30 and cutting element 1040 can be detachable from each other and detachable from the internal shaft 1025.

Referring now to FIG. 12, the bone removal device 1005 is mounted to one of the anchoring devices 60, 80 using the coupling 1050 and receptacles (not shown) on the anchoring devices 60, 80. The receptacles can be any feature that receives fasteners 1055 on the coupling 1050, thereby attaching the bone removal device 1005 to the anchoring device.

The anchoring devices 60, 80 allow for manipulation of the bone removal device 1005 such that the cutting element 1040 can be positioned adjacent to one of adjacent vertebral endplates 14, 16, and moved into a proximity with the selected endplate sufficient to permit shaping of the endplate with the cutting element. Positioning of the cutting element 1040 may be established with known offsets and may be verified with fluoroscopic or other imaging techniques.

In operation, a power source (not shown) is provided to the bone removal device 1005 to drive the internal shaft 1025. The internal shaft 1025 may directly or indirectly drive the contour 1030, thereby actuating the cutting element 1040 and causing the cutting edge 1042 to rotate around the contour 1030. For example, the internal shaft 1025 may be connected to a connecting portion of the cutting element as described above with respect to FIGS. 2A and 2B. In such an example, actuation of the internal shaft to cause rotation of the connecting portion will cause rotation of other portions of the cutting element, including causing the cutting edge to rotate around the contour.

In certain examples, the internal shaft is actuated to rotate in a clockwise or counterclockwise direction, and the cutting element 1040 will also rotate in a clockwise or counterclockwise direction along the same axis of rotation as the internal shaft 1025. The actuated cutting element shapes the vertebral endplate to which it is adjacent. The contour 1030 and cutting element 1040 are shaped such that the profile created in the vertebral endplate corresponds at least in part to the profile of the selected intervertebral prosthesis or fusion device 18.

After the first endplate is prepared, the bone removal device may be mounted to the other of the anchoring devices 60, 80 with the cutting element 1040 positioned adjacent to the other of the vertebral endplates 14, 16. The bone removal device 1005 is again powered, this time to shape the second endplate. In certain examples, the same contour and cutting element are used to shape the first endplate will be used to shape the second endplate. In other examples, a different contour and cutting element are used, which may have a different profile that corresponds at least in another part to the profile of the implant.

In this exemplary embodiment, the anchoring devices 60, 80 remain fixedly aligned to the vertebral bodies and rotatably connected to distracting arms 46. As such, the vertebral bodies 14, 16 may be permitted to rotate independently of each other and therefore, permit each of the vertebral bodies to be shaped independently.

After the vertebral endplates are prepared, the bone removal device 1005 may be removed from the anchoring device 60 or 80 in preparation for implanting an intervertebral prosthesis. With the cutting instrumentation removed, the intervertebral prosthesis may be inserted into the prepared space using any of a variety of insertion methods. In some embodiments, the anchoring devices 60, 80 may be used to guide prosthesis insertion instrumentation. After the prosthesis is implanted, the tension on the distractor assembly 40 may be released. The anchoring fixtures 92, 94 may be removed form the vertebral bodies 16, 14 respectively, permitting the distractor assembly 40 to be removed. With all instrumentation removed from the disc site, the wound may be closed.

The distractor assembly 40 and anchoring devices 60, 80 described herein are merely exemplary embodiments that may be used with a bone removal device 5, 1005 described herein. In alternative embodiments, any assembly suitable for providing access to a space into which a prosthesis will be implanted, and for providing any alignment or anchoring necessary to prepare the space for use of the bone removal device described herein is suitable.

With a suitably shaped and sized contour and cutting element, a bone removal device as described herein is useful in the cervical, thoracic, and lumbar spine from anterior to the transverse processes of the vertebrae, lateral or anterolateral in the thoracic and lumbar spines, or from posterior in the lumbar spine.

The invention has been described above with respect to certain specific embodiments thereof. Those of skill in the art will understand that variations from these specific embodiments that ate within the spirit of the invention will fall within the scope of the appended claims and equivalents thereto.

What is claimed is:

1. A device comprising:
a contour having a predetermined profile corresponding at least in part to the profile of an implant; and
a cutting element rotatably attached to the contour, which cutting element has a cutting edge having a profile corresponding to the contour profile.

2. The device of claim 1 further comprising:
a neck terminating the contour at a distal end;
a bore extending through the contour and having an exit at the distal end of the contour through the neck;
a bore mating portion of the cutting element, which rotatably extends through the bore and exits the bore at the neck; and
a connecting portion of the cutting element, which terminates the bore mating portion at a distal end of the bore mating portion.

3. The device of claim 2 further comprising:
an annular recess formed on the neck; and
a neck mating portion of the cutting element rotatably residing within the annular recess.

4. The device of claim 2 further comprising:
a shaft connected to the connecting portion; and
a power source operable to actuate the shaft and cause rotation of the connecting portion, the neck mating portion, the bore mating portion and the cutting edge.

5. The device of claim 1 further comprising;
an interior shaft connected to the contour; and
an exterior shaft through which the shaft extends.

6. The device of claim 5 further comprising:
a coupling attached to the exterior shaft.

7. The device of claim 6 wherein the coupling comprises fasteners operable to attach the device to one or more other devices.

8. The device of claim 1 wherein the cutting element comprises a wire or a metal blade.

9. The device of claim 1 wherein the cutting element is formed from at least one material selected from the group consisting of nitinol, titanium alloy, stainless steel, plastic, polyester, and polyethylene.

10. The device of claim 1 wherein the predetermined profile of the contour has a shape selected from the group consisting of egg-shaped, parabolic and double-hump.

11. A system for preparing a site comprising:
an access instrument operable to provide access to the site; and
a bone removal device, which bone removal device comprises a contour and a cutting element attached to the contour, which contour has a profile corresponding at least in part to the profile of an implant to be inserted at the site, and which cutting element has a cutting edge having a profile corresponding to the contour profile.

12. The system of claim 11 wherein:
the site comprises a space between a first vertebrae and a second vertebrae, and wherein,
the access instrument comprises:
a first distraction arm;
a second distraction arm;
a first anchoring device attached to both the first distraction arm and the first vertebra; and
a second anchoring device attached to both the second distraction arm and the second vertebrae.

13. The system of claim 12 wherein the first anchoring device moves independently of the second anchoring device.

14. The system of claim 13 wherein the movement of the first anchoring device is at least one of: movement in a sagittal plane, movement in a transverse plane, pivotal movement, and linear movement in an anterior-posterior direction.

15. A system for preparing a site comprising:
means for providing access to the site; and
means for shaping a surface at the site to have a profile corresponding at least in part to the profile of a device to be inserted into contact with the surface.

16. A method of preparing a space between first and second vertebral bodies to receive an implant, the method comprising:
attaching first and second anchoring devices to the first and second vertebral bodies, respectively;
attaching a distraction assembly to the first and second anchoring devices, wherein a first arm of the distraction assembly is attached to the first anchoring device and a second arm of the distraction assembly is attached to the second anchoring device; and
attaching a bone removal device to the first distractor arm, which bone removal device comprises a contour and a cutting element attached to the contour, which contour has a profile corresponding at least in part to the profile of the implant, and which cutting element has a cutting edge having a profile corresponding to the contour profile; and
shaping a first endplate of the first vertebral body with the cutting element.

17. The method of claim 16 further comprising:
moving the first and second arms of the distraction assembly, in parallel, relative to one another, prior to shaping the first endplate.

18. The method of claim 16 further comprising independently moving the first and second anchoring devices relative to the first and second arms, respectively.

19. The method of claim 16 further comprising
removing the bone removal device from the first distractor arm;
replacing the bone removal device on the second distractor arm; and
shaping a second endplate of a second vertebral body.

20. The method of claim 16 wherein
the bone removal device further comprises a shaft, and the contour is removably attached to the shaft, and wherein the method further comprises,
detaching the contour, with the cutting element attached to the contour, from the shaft; and
replacing the contour with a second contour having a second cutting element attached thereto.

21. The method of claim 20 wherein the second contour has a profile different than the profile of the detached contour, and wherein the second cutting element has a cutting edge having a profile corresponding to the second contour profile.

22. A method of preparing a site between first and second vertebral bodies to receive an implant, the method comprising:
creating space between first and second vertebral bodies; and
shaping a first endplate of the first vertebral body with a bone removal device comprising a contour and a cutting element attached to the contour, which contour has a profile corresponding at least in part to the profile of the implant, and which cutting element has a cutting edge having a profile corresponding to the contour profile.

23. The method of claim 22 further comprising:
creating the space between the first and second vertebral body with at least one of a distractor, a spreader, and a distraction assembly.

24. The method of claim 22 further comprising
shaping a second endplate of a second vertebral body with the bone removal device.

25. The method of claim 24 wherein
the bone removal device further comprises a shaft, and the contour is removably attached to the shaft, and wherein the method further comprises,
detaching the contour, with the cutting element attached to the contour, from the shaft; and
replacing the contour with a second contour having a second cutting element attached thereto.

26. The method of claim 25 wherein the second contour has a profile different than the profile of the detached contour, and wherein the second cutting element has a second cutting edge having a profile corresponding to the second contour profile.
